(19) United States
(12) Patent Application Publication
(10) Pub. No.: US 2007/0185215 A1
(43) Pub. Date: Aug. 9, 2007

(54) Pharmaceutical composition containing obovatol as an active ingredient for the prevention and treatment of neurodegenerative diseases

(76) Inventors: Byoung-Mog Kwon, Yuseong-ku (KR); Kwang-Hee Son, Yuseong-ku (KR); Dong Cho Han, Yuseong-ku (KR); Ji-Min Lee, Pohang-si (KR); Kyoungho Suk, Suseong-ku (KR); Su Hyung Hong, Suseong-ku (KR); Jiyeon Ock, Euyryeong-gun (KR)

Correspondence Address: LUCAS & MERCANTI, LLP, 475 PARK AVENUE SOUTH, 15TH FLOOR, NEW YORK, NY, 10016, US

(21) Appl. No.: 11/401,341

(22) Filed: Apr. 10, 2006

(30) Foreign Application Priority Data
Feb. 8, 2006 (KR) 10-2006-0012060

(51) Int. Cl. A61K 31/075

(52) U.S. Cl. 514/720

(57) ABSTRACT

Disclosed herein a pharmaceutical composition, comprising obovatol as an active ingredient, for the prevention and treatment of neurodegenerative diseases. Having superior inhibitory activity against the production of neurotoxic nitric oxides, the obovatol isolated and purified from Magnoliaceae can be used as active ingredient for a pharmaceutical composition or a neuroprotective agent for the prevention and treatment of neurodegenerative diseases, such as Alzheimer's disease, Parkinson's disease, and multiple sclerosis.

### BACKGROUND OF THE INVENTION

1. Field of the Invention

The present invention relates to a pharmaceutical composition, containing obovatol as an active ingredient, for the prevention and treatment of neurodegenerative diseases.

2. Description of the Prior Art

With the rapid global increase in an aging population, the morbidity of neurodegenerative diseases (NDDs) is expected to overtake that of cancer, which is the most important cause of death after cardiovascular diseases. Accordingly, the market for therapies for NDDs, such as Alzheimer's disease, Parkinson's diseases, etc., has grown by 20% every year since 2000. Recent reports have disclosed that the market for Alzheimer's disease therapy has sharply grown from eight hundred million dollars in 2000 to 1.7 billion dollars in 2002 and 3.3 billion dollars in 2004. Also, the market for Parkinson's disease therapy is reported to grow gradually, but steadily, reaching 1.6 billion dollars in 2000, 1.9 billion dollars in 2002 and 2.5 billion in 2004. As proven by market expansion, more extensive attention has been paid to NDDs.

NDDs, of which Alzheimer's disease, Parkinson's disease, and multiple sclerosis are representative, are caused by the gradual deterioration of neurons and feature problems with cognition or movement, leading to the death of the patients thereof. NDDs are also characterized by the increase of morbidity with aging. The correlation between brain aging and NDDS, although not explicitly revealed so far, is partially based on the common properties therebetween, including loss of nerve cells and reduction in brain volume. Therefore, study on NDDs should be followed by study on brain aging.

In response to abnormal stimulation caused by infection, trauma, internal tissue injury, etc., the living body reacts so as to control or destroy the factor causing the abnormal stimulation and starts to conduct a recovery process by means of the inherent immune system so as to prevent subsequent tissue injury and restore normal functionality. This biological response is called “inflammatory response”. Immune cells activated during the inflammatory response may often cause further tissue damage while responding to the injured site. Thus, accurate control and balance during the overall response process are very important. Unless the balance of inflammatory response is suitably maintained, tissue injury, rather than tissue protection or recovery, may occur, resulting in inflammatory diseases. The inflammatory response within the central nervous system is led by neuroglia. Recent extensive research results have marked neuroglia as a key player in inflammatory responses in the central nervous system. Indeed, neuroglia are known to produce inflammatory mediators such as cytokines, nitric oxide (NO), prostaglandins, etc., in various pathogenic conditions of the central nervous system, thereby either healing injured sites or causing further nerve tissue injury. The activation of neuroglia in response to central nervous system inflammation is found throughout all of the brain tissues, and the proliferation and apoptosis thereof has been observed. Additionally, the recent report explains that the formation of cholesterol for synaptogenesis is accomplished by the neuroglia, which regulate the chemical and electrical environment of the brain, especially the fluid surrounding neurons and their synapses, providing a clue for the treatment of Alzheimer's disease.

Nerve tissues are comprised of neurons and neuroglia. Neurons are responsible for the substantial functioning of nerve tissues. Located between blood vessels and neurons, neuroglia function to provide substances necessary for neurons in the brain and the spinal cord. Some neuroglia function primarily as physical support for neurons. Others regulate the external chemical environment of neurons by nutrition supply, waste removal, phagocytic action, etc., thereby preventing pathogens, such as bacteria or toxins, from penetrating into nerve cells.

The neuroglial cells are classified into three categories:

Astrocytes, the most abundant type of neuroglia, are individually star shaped, with multiple radial projections. These cells have vascular end feet and ampullae positioned at the end of the projections, which are in contact with capillary vessels, functioning to transmit metabolites from vessels to neurons. There are two varieties of astrocytes: protoplasmic and fibrous astrocytes.

Oligodendrocytes, another type of neuroglia, have a small number of undifferentiated projections and are involved in myelin sheath formation, functioning like Schwann cells in the peripheral nervous system.

Microglia account for 10˜12% of the count of neuroglia in the central nervous system and were first discovered in 1932 through a morphological study and tissue staining method by Rio-Hortega. Microglia act like phagocytes in the central nervous system (CNS), cleaning up CNS debris, degenerated nerve cells, exogenous materials, etc. Microglia are regarded as close cousins of other phagocytic cells including macrophages, playing numerous important roles in protecting the nervous system, including transportation, destruction, removal, and scavenging of pathogenic metabolites. In addition, the macrophage-like cells express cell surface antigens, which are the most distinct from other neuroglial cells (astrocytes and oligodendrocytes). Monocytes, known as the progenitor cells of microglia, migrate into the central nervous system during development, and differentiate into microglia therein. Microglia primarily defend the CNS when microbial infection or injury occurs. Once activated in response to antigenic stimulation, microglia proliferate and migrate to the site of injury where they perform phagocytosis on the infecting microbes or digest injured neuron debris. During the performance of their own functions, that is, defense of the central nervous system, microglia produce tumor necrosis factor(TNF)-α, interleukin(IL)-1β, reactive oxygen species (ROS) and nitrogen compounds. However, when these inflammatory materials are secreted excessively or microglia are maintained in an active state for a long time, a serious side effect, nerve tissue injury, results. Recent studies indicate that the hyperactivation of microglia is associated with trauma-induced or ischemic neuronal injury as well as neurodegenerative diseases, such as Alzheimer's disease, Parkinson's disease, etc. [Microglia as mediators of inflammatory and degenerative diseases. Gonzalez-Scarano, F and Baltuch, G. Annu. Rev. Neurosci. 22, 219-40, 1999]. On the basis of the microglial mechanism of inflammatory and degenerative diseases, therapies have been developed for suppressing activated microglia or inhibiting the activity of the inflammatory materials secreted from the activated microglia.

Also, there are other types of neuroglial cells, including ependymal cells, Schwann cells and satellite cells. Ependymal cells line the cavities of the central nervous system and beat their cilia surrounding capillary vessels to help circulate the cerebrospinal fluid. Similar in function to oligodendrocytes, Schwann cells provide myelination to axons in the peripheral nervous system and thus insulate the nerves from the external environment. Satellite cells are small flat cells that line the exterior surface of peripheral nervous system neurons and help regulate the external chemical environment.

Consequently, neuroinflammation, which is primarily-induced by the activation of microglia, is a main cause of various neuronal injuries, thus the inhibition of the inflammatory activation of microglia can be a strategy for preventing neuronal injuries, leading to the development of drugs that are preventive and curative of neurodegenerative diseases.

Leading to the present invention, intensive and thorough research, conducted by the present inventors, into the suppression of neuronal injuries induced by the inflammatory activation of microglia, resulted in the surprising finding that obovatol has inhibitory activity against the microglial activation-induced production of nitric oxides, and thus can be useful for the prevention and treatment of neurodegenerative diseases.

### SUMMARY OF THE INVENTION

Therefore, it is an object of the present invention to provide a pharmaceutical composition for the prevention and treatment of neurodegenerative diseases, containing obovatol as an active ingredient.

It is another object of the present invention to provide a neuroprotective agent containing obovatol as an active ingredient.

It is a further object of the present invention to provide a method for preventing and treating neurodegenerative diseases using a composition containing obovatol as an active ingredient.

### DESCRIPTION OF THE PREFERRED EMBODIMENT

In accordance with an aspect of the present invention, a pharmaceutical composition comprising obovatol as an active ingredient is provided for the prevention and treatment of neurodegenerative diseases.

In accordance with another aspect of the present invention, a neuroprotective agent comprising obovatol as an active ingredient is provided.

In accordance with a further aspect of the present invention, a method for preventing and treating neurodegenerative diseases is provided, in which the composition comprising obovatol as an active ingredient is used.

Hereinafter, the present invention is described in detail.

Based on obovatol, represented by the following formula 1, the present invention pertains to a composition and a neuroprotective for the prevention and treatment of neurodegenerative diseases. Obovatol, as an active ingredient in the present invention, is preferably isolated and purified from silver magnolia (Magnolia obovata Thunberg).

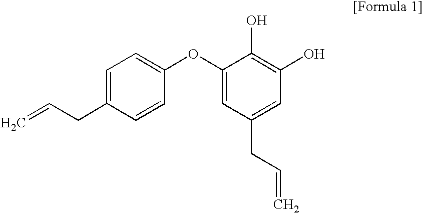

Silver magnolia [Magnolia obovata Thunberg], belonging to the Magnoliaceae family, is a deciduous tree, which grows perennially, and is known to have an effect of dampness elimination by promoting movement of the vital energy and antiasthmatics. In herbal medicine, dried cortexes of silver magnolia have been used as an agent for strengthen the stomach and digestion, a purgation, an antitussive, and a expectorant to treat aromatic strengthen the stomach, stomachache, physconia, acute enteritis, cough, etc. and as pharmaceutical ingredient of Wiryeongtang, Kwahyangjeonggisan, Soseunggitang, Bakhahubaktang, Pyeong-wisan, Mazainhwan, etc. This plant is known to contain various useful compounds including β-eudesmol, magnolol, honokiol, magnocurarine, and tannin. Among them, magnolol and honokiol have been proven to show more safe and potent antibiotic activity than do conventional antibiotics such as chlorohexidine (Korean Pat. Laid-Open Publication No. 1992-16516). Much has been reported about the various physiological activities of these ingredients of Magnoliaceae as an antibacterial agent in Korean Pat. Laid-Open Publication No. 1992-16516, a composition for an anticancer in Korean Pat. Laid-Open Publication No. 1999-10867, and a composition for an antituberculosis in Korean Pat. Laid-Open Publication No. 1988-09390. Additionally, the ingredients of Magnoliaceae are used for the treatment of AIDS (Korean Pat. Laid-Open Publication No. 1991-09170) and hair loss (Korean Pat. Laid-Open Publication No. 2000-51040) and are found to show smooth muscle relaxation, anti-Helicobacter pylori activity, an anti-allergic effect, an anti-cancer effect, and an inhibitory effect on ischemic reperfusion-induced small intestine injury. However, nowhere has the neuroprotective effect of obovatol from magnolia been mentioned in the previous literature.

Obovatol according to the present invention may be extracted from Magnoliaceae in a manner well known in the art. Synthetic or commercially available obovatol is also useful in the present invention. Preferably, obovatol may be extracted from Magnoliaceae in accordance with the method of the present invention, as follows.

In an embodiment of the present invention, obovatol may be isolated and purified by:

(a) extracting leaves, fruit, and/or bark of silver magnolia using an organic solvent; and

(b) fractionating the extract by means of silica gel chromatography to separate compounds.

In the step(a), leaves, fruit, bark, or all of these are extracted with an organic solvent.

The leaves, fruit and bark, whether just harvested or on the market, may be used without limitation. Before extraction, they are cleaned and dried. Dried silver magnolia leaves, fruit, bark, or combinations thereof are chopped into suitable sizes and immersed in a proper amount of an organic solvent, preferably methanol. After standing for 24˜50 hours at room temperature, the solution is allowed to go through a filter. Optionally, the filtrate may be concentrated or freeze-dried.

Next, in the step(b), the extract from leaves, bark and fruit in organic solvent is fractionated using silica gel chromatography to separate compounds and purify obvatol.

For the silica gel chromatography, methylene chloride is preferably used as a solvent while a mixture of 90:10 to 80:20 ethylacetate and hexane is preferably used as a mobile phase. Separated compounds may be allowed to undergo a typical purification step using C₁₈ column chromatography.

In accordance with the present invention, obovatol is found to reduce the production of nitric oxides, which are neurotoxic materials produced from the microglia activated by inflammatory stimuli, such as LPS (lipopolysaccharide), and to show no toxicity in cultured mammalian cells or experimental animals, so that it can be used as an ingredient for an effective pharmaceutical composition or a neuroprotective agent for the prevention and treatment of neurodegenerative diseases, such as Alzheimer's disease, Parkinson's disease, and multiple sclerosis.

The pharmaceutical composition in accordance with the present invention may be in a dosage form suitable for oral or non-oral administration. In order to prepare the pharmaceutical composition in accordance with the present invention, obovatol may be formulated with a typical vehicle, like a filler, a thickening agent, a binder, a wetting agent, a disintegrant, a surfactant, a diluent, an expedient, etc. Solid formulations suitable for oral administration include tablets, pills, powders, granules, capsules, etc. and may be formulated with at least one expedient, such as starch, calcium carbonate, sucrose, lactose, gelatin, etc. In addition to these, a lubricant, such as magnesium stearate talc, etc., may be contained in the composition of the present invention.

Liquid formulations suitable for oral administration, exemplified by suspensions, peroral solutions, emulsions, syrup, etc., may comprise various expedients, such as wetting agents, sweetening agents, flavors, and preservatives, as well as simple diluents such as water, liquid paraffin, etc.

As for non-oral formulations, sterile aqueous solutions, non-aqueous solutions, suspensions, emulsions, lyophilized injections, suppositories, etc. may be exemplary. Non-aqueous solutions and suspensions may contain propylene glycol, polyethylene glycol, vegetable oil such as olive oil, and injectable esters such as ethylolate. As a base for suppositories, witepsol, Macrogol, Tween 61, cacao butter, laurinic acid, or glycerogeratin may be used.

Depending on patients' age, sex, and weight, the compound according to the present invention may be administered in a total dosage from 50 to 100 mg/day, and preferably in a dosage from 50 to 80 mg/day. This dosage may be administered once a day or in a partitioned manner several times a day, and preferably once or three times a day.

For preventing and treating neurodegenerative diseases, the composition of the present invention may be used alone or in combination with surgical operation, hormonal therapy, other drugs, or biological response regulators.

A better understanding of the present invention may be obtained through the following examples which are set forth to illustrate, but are not to be construed as the limit of the present invention.

### EXAMPLE 1

### Preparation of Obovatol

### 1-1 Extraction, Isolation and Purification of Obovatol from Magnolia

2 kg of Magnolia leaves (taken from trees naturally growing in the central region of Korea) were cut into pieces, and were put in a vessel. Then 5 liters of methanol was added thereto and allowed to stand for 48 hours at room temperature. After stirred, using filter paper, solids were filtered out. The liquid phase was collected and concentrated in a vacuum, and the concentrate was dissolved in methanol. The organic layers containing active substance were collected and concentrated in vacuo to yield 120 g of magnolia leaf extract.

The concentrate was dissolved in methylene chloride and placed on a silica gel (Merck, Art No. 9385) to adsorb the active substance thereonto. Silica gel column chromatography was conducted with an ethylacetate-hexane gradient varying from 90:10 to 80:20, so as to yield active fractions. After the adsorption of the fractions onto a C₁₈ column, elution with methanol and water led to the partial purification of the active substance, which was further purified to completion through silica gel column chromatography to produce 25 g of a pure compound.

### 1-2 Structural Analysis of the Purified Compound

The compound purified in Example 1-1 was analyzed for its molecular weight and molecular formula by UV absorbance, IR (infrared) absorbance and high resolution mass spectrometry.

In this regard, a UV-265 spectrophotometer (Shimadzu) was used for UV absorbance analysis, a Digilab Division FTS-80 spectrophotometer (Bio-Rad) for IR absorbance analysis, and high resolution VG70-SEQ mass spectrometry (MS) for the determination of molecular weight and molecular formula. Also, ¹H and ¹³C-NMR spectra were obtained using a nuclear magnetic resonator (Varian 300 MHz, 500 MHz NMR) and analyzed to determine the structure of the compound.

Physical and chemical properties are given in Table 1 for obovatol.

TABLE 1
Obovatol

| Appearance | | Pale green liquid |
|---|---|---|
| Empirical Molecular Formula | | C₁₈H₁₈O₃ |
| Mw | | 282 |
| Solubility | Soluble | Alcohol, DMSO |
| | Insoluble | Hexane, water |

| | |
|---|---|
| NMR data | ¹H-NMR(300MHz, CDCl₃) δ=6.28(d, |
| | J=1.8Hz), 6.56(d, J=1.8Hz), |
| | 3.18(d, J=6.6Hz), 5.97(m), 5.09(m), |
| | 6.93(d, J=4.3Hz), 7.14(d, |
| | J=4.3Hz), 3.36(d, J=6.6Hz). |

As shown in Table 1, the compound isolated and purified in Example 1-1 was identified to be obovatol.

### EXPERIMENTAL EXAMPLE 1

### Assay for Inhibitory Activity against Activation of Microglia

To examine the effect of the obovatol prepared in the above examples on nervous system, the following test was performed.

The BV-2 murine microglial cell line, obtained from Prof. Eui-Ju, Choi, School of Life Sciences and Technology in Korea University, Korea, was treated with 100 ng/ml of LPS (lipopolysaccharide), known to activate microglial cells, in the presence of 1 μg/ml or 10 μg/ml obovatol for 24 hours, and then the nitric oxides secreted into the culture media were quantitatively analyzed using a Griess reaction method to determine the activity of the microglia. In detail, 50 μl of the microglial culture medium was reacted with 50 μl of the Griess reagent [1% sulfanilamide/0.1% naphthylethylene diamine dihydrochloride/2% phosphoric acid] at room temperature for 10 min, followed by measuring absorbance at 540 nm with the aid of a microplate reader (Anthos Labtec Instruments GmbH Salzburg, Austria). For comparison, magnolol, known to have a neuroprotective effect, was also tested in the same manner. Using NaNO₂ as a standard material, inhibition rates of the test materials were calculated for the production of nitric oxides. Also, MTT assay was conducted to measure cytotoxicity in order to determine whether or not the inhibitory activity against microglial activity was attributed to cytotoxicity.

Rates of inhibition of the production of nitric oxide are given in Table 2, below.

TABLE 2
Inhibition Rates (%) of Nitric Oxide Production

Concentration (μg/ml)

| Compounds | 1 | 10 |
|---|---|---|
| Obovatol | 29.8 | 83.3 |
| Magnolol | 13.1 | 59.0 |

As seen in Table 2, obovatol inhibited the microglial production of nitric oxides in a dose-dependent manner with superior effectiveness as compared to magnolol.

Obovatol did not exhibit cytotoxicity in concentrations as high as 30˜50 μg/ml, as measured by MTT assay, indicating that the inhibitory activity of nitric oxide production of microglia was not due to cytotoxicity.

Therefore, obovatol prepared from Magnoliaceae according to the present invention was proven to inhibit microglial neurotoxicity.

### EXPERIMENTAL EXAMPLE 2

### Acute Oral Toxicity Assay in Rats

Obovatol, prepared from Magnoliaceae, was assayed for acute toxicity in experimental animals as follows.

Using six-week-old specific pathogen-free (SPF) SD rats, an acute toxicity assay was conducted. The rats were divided into groups of two rats. After being dissolved in injectable saline, the obovatol, obtained in Example 1, was orally administered once in a dosage of 1 g/kg/ml to the rat groups. Afterwards, observations were made of the death, clinical symptoms, and weight changes of the animals, and serological and serobiochemical assays were conducted. Also, an autopsy was carried out to examine abnormalities of the abdominal and thoracic organs with the naked eye.

None of the animals to which the compound of interest was administered exhibited noticeable clinical symptoms or died. Cytotoxicity was not observed in the weight change, serological assay, serobiochemical assay, or autopsy observations for the animals administered with the compounds of the present invention.

Causing no toxic effects up to a dose of 500 mg/kg, the obovatol, purified according to the present invention, was determined to have an oral lethal dose (LD₅₀) of 500 mg/kg, and thus was regarded as safe.

### FORMULATION EXAMPLE 1

### Preparation of Pharmaceutical Formula

1-1: Preparation of Powder

| | |
|---|---|
| Ovobatol | 2 g |
| Lactose | 1 g |

The above ingredients were mixed and charged in an airtight sac to afford a powder agent.

1-2: Preparation of Tablet

| | |
|---|---|
| Ovobatol | 100 mg |
| Corn Starch | 100 mg |
| Lactose | 100 mg |
| Magnesium Stearate | 2 mg |

The above ingredients were mixed and formed into a tablet using a general tabletting method.

1-3: Preparation of Capsule

| | |
|---|---|
| Ovobatol | 100 mg |
| Corn starch | 100 mg |
| Lactose | 100 mg |
| Magnesium stearate | 2 mg |

These ingredients were mixed and filled into a gelatin capsule according to a typical procedure, so as to give a capsule agent.

1-4: Preparation of Injection

| | |
|---|---|
| Ovobatol | 10 μg/ml |
| dil. HCl BP | Added to achieve pH 3.5 |
| Injectable NaCl BP | 1 ml at the most |

A solution of obovatol in a proper volume of injectable sodium chloride BP was adjusted to pH 3.5 with diluted hydrochloric acid BP, and its volume was adjusted with injectable sodium chloride BP. After being sufficiently mixed, the solution was filled in a 5 ml type I ampoule made from transparent glass, which was then melted so that the solution was packaged with a small amount of air. An injection was obtained by autoclaving at 120° C. for 15 min or longer.

As described hereinbefore, the obovatol is able to effectively inhibit the production of nitric oxides from activated microglia without cytotoxicity, and therefore can be used in a pharmaceutical composition or a neuroprotective agent for the prevention and treatment of neurodegenerative diseases, such as Alzheimer's disease, Parkinson's disease, and multiple sclerosis.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A pharmaceutical composition for the prevention and treatment of neurodegenerative diseases, comprising obovatol as an active ingredient.

2. The pharmaceutical composition as defined in claim 1, wherein the obovatol is isolated and purified from a silver magnolia.

3. The pharmaceutical composition as defined in claim 2, wherein the obovatol is prepared by:
(a) extracting leaves, fruit, and/or bark of silver magnolia using an organic solvent; and
(b) fractionating the extract by means of silica gel chromatography to separate compounds.

4. The pharmaceutical composition as defined in claim 3, wherein the organic solvent is methanol.

5. The pharmaceutical composition as defined in claim 3, wherein the silica chromatography is conducted using methylene chloride as a solvent.

6. The pharmaceutical composition as defined in claim 3, wherein the silica gel chromatography is conduced using a mixture of ethyl acetate and hexane as a mobile phase.

7. The pharmaceutical composition as defined in claim 6, wherein the mixture contains ethyl acetate and hexane at a volume ratio from 90:10 to 80:20.

8. The pharmaceutical composition as defined in one of claims 1 to 7, wherein the neurodegenerative diseases are selected from among Alzheimer's disease, Parkinson's disease, and multiple sclerosis.

9. The pharmaceutical composition as defined in claim 8, wherein the pharmaceutical composition is in a dosage form suitable for oral administration or non-oral administration.

10. The pharmaceutical composition as defined in claim 9, wherein the dosage form for oral administration is a tablet, a pill, a powder, a granule, a syrup, a liquid, a suspension, an emulsion, or a capsule.

11. The pharmaceutical composition as defined in claim 9, wherein the dosage form for non-oral administration is an injection, a rectal suppository, or a transdermal agent.

12. A method for preventing and treating neurodegenerative diseases, comprising administering the composition of one of claims 1 to 11 in a therapeutically effective dosage.

13. The method as defined in claim 12, wherein the method is used alone or in combination with surgical operation, hormonal therapy, other drugs, or biological response regulators.

14. A neuroprotective agent, comprising obovatol as an active ingredient.
